(19) United States
(12) Patent Application Publication
(10) Pub. No.: US 2012/0178900 A1
(43) Pub. Date: Jul. 12, 2012

(54) CHROMATOGRAPHIC PROCESSES AND PURIFIED COMPOUNDS THEREOF

(75) Inventors: Nitesh Dave, Bangalore (IN); Krishana Chaitanya Gulla, Indore (IN); Sundaresh Shankar, Bangalore (IN); Harish Iyer, Bangalore (IN)

(73) Assignee: BIOCON LIMITED, Bangalore, Karnataka (IN)

(21) Appl. No.: 13/390,094

(22) PCT Filed: Aug. 9, 2010
(86) PCT No.: PCT/IB2010/053589

§ 371 Date: Feb. 10, 2012

(30) Foreign Application Priority Data
Aug. 11, 2009 (IN) 01908/CHE/2009

(51) Int. Cl. C07K 1/20; C07K 7/16; C07K 7/50; C07K 14/62

(52) U.S. Cl. 530/305; 530/315; 530/317; 530/303

(57) ABSTRACT

The present disclosure demonstrates the utility of ion pairing agents in the preparative scale of purification. More particularly, the disclosure relates to the usage of ion pairing agents in RP preparative linear chromatography enabling high purity of the desired end product. The disclosure shows that ion-pairing agents have dramatic effect on desired purity of polypeptides.

### TECHNICAL FIELD

The present disclosure demonstrates the utility of ion pairing agents in the preparative scale of purification. More particularly, the disclosure relates to the usage of ion pairing agents in reverse phase preparative chromatography enabling high purity of the desired end product. The disclosure shows that ion-pairing agents have dramatic effect on desired purity of polypeptides.

### BACKGROUND AND PRIOR ART OF THE DISCLOSURE

A number of different chromatographic procedures are applied to obtain the desired end result with respect to purity and yield. Reverse-phase chromatography is one of the most powerful methods of purification employed. Reverse phase liquid chromatography (“RP-LC”) and reverse phase high-performance liquid chromatography (“RP-HPLC”) are commonly used to purify molecules such as peptides and proteins , produced by either synthetic or recombinant methods. RP-LC and RP-HPLC methods can efficiently separate closely related impurities and have been used to purify many diverse molecules (Lee et al., “Preparative HPLC,” 8^{th} Biotechnology Symposium, Pt. 1, 593-610 (1988)). Further, RP-LC and RP-HPLC have been successfully used to purify molecules, particularly; proteins on an industrial scale (Olsen et al., 1994, J. Chromatog. A, 675, 101).

The usage of ion pairing agents such as triethylamine (TEA), Trifluoro acetic acid (TFA), Hexane sulfonic acid (HSA) etc in the analytical method development and its effect on protein peak resolution is well known Shayne Cox Gad; Handbook of Pharmaceutical Biotechnology. An ion pairing agent adsorbs to the stationary phase via its hydrocarbon chain creating an electrical double layer which imposes an electric potential on the surface of the stationary phase. Due to this, the protein/peptide molecules experience both ion exchange with the electrolyte ions of the mobile phase and also the adsorption effect with the reverse phase stationary phase (Frederick F. Cantwell et al., 1984, Journal of Pharmaceutical and Biomedical Analysis, Volume 2, Pages 153-164).

The instant disclosure relates to usage of ion pairing agents in the preparative scale of purification of proteins and peptides.

More specifically this disclosure relates to the use of HSA and TEA in RP-HPLC preparative linear chromatography.

The ion pairing agents used in RP-HPLC essentially contain long hydrocarbon chain with an ionisable group. These molecules, in the mobile phase ion pair with the surface charges of the protein/peptide. Due to this ion pairing, the hydrophobicity of the peptide/protein increases which is attributed to the hydrocarbon chain of the ion pairing agent. This interaction depends on the surface charges. Different proteins of a mixture have different surface charges, and hence, bind differentially to the stationary phase which improves the resolution between the protein peaks. The adsorbed ion pairing agents impart a charge specific to its functional group on the surface of the stationary phase. This repels the like charges, thus, changing the selectivity of various proteins in a mixture.

It has been observed in conventional chromatography that as the loading increases on the column the resolution decreases between the impurities and desired protein of interest. The protein bands during elution tend to merge affecting the performance of the chromatography in terms of purity of the preparation and yield. The instant disclosure circumvents this problem which is a key to manufacturability of proteins. Higher loading on the column beyond the protein injected on the column for analytical detection, is crucial for process development and dictates the cost and yield of a given process. The instant disclosure shows that ion pairing agents have dramatic effect in the purity of proteins even after the protein loading was increased. The instant disclosure demonstrates the use of ion pairing in improving yield of the chromatographic step.

### OBJECTIVES OF THE DISCLOSURE

The main objective of the present disclosure is to obtain a chromatographic process for the purification of polypeptides from a mixture.

Another main objective of the present disclosure is to obtain, Insulin analogue such as Aspart, Glargine and Lispro.

Yet another main objective of the present disclosure is to obtain purified polypeptides such as Atosiban and Eptifibatide.

### SUMMARY OF THE DISCLOSURE

Accordingly, the present disclosure relates to a chromatographic process for purification of polypeptide from a mixture having at least one related impurity, said process comprising step of employing RP-HPLC with an ion paring agent having concentration ranging from about 0.01% to about 2% in combination with organic modifier having concentration ranging from about 5% to about 85%; a chromatographic process for purification of polypeptide from a mixture having at least one related impurity, said process comprising steps of a) packing RP-HPLC column with silica (C₄—C₁₈) based resin, equilibrated with about 5% to about 85% organic modifier, b) loading the polypeptide mixture on the column at a flow rate of about 180 cm/hr to about 360 cm/hr, c) washing the column with an ion pairing agent having concentration ranging from about 0.05% to about 1% in combination with the organic modifier having concentration ranging from about 5% to about 85%, and d) performing a linear gradient of about 10% to about 70% for eluting the purified polypeptide from the column; an Insulin analogue obtained by a process as stated above with purity ranging from about 90% to about 100%; purified Aspart purified with a purity of at least 98%; purified Glargine purified with a purity of at least 99%; purified Lispro purified with a purity of at least 97%; a polypeptide obtained by a process as stated above with purity ranging from about 90% to about 100%, purified Atosiban purified with a purity of at least 99.14%; and purified Eptifibatide purified with a purity of at least 94%.

### DETAILED DESCRIPTION OF THE DISCLOSURE

The present disclosure relates to a chromatographic process for purification of polypeptide from a mixture having at least one related impurity, said process comprising step of employing RP-HPLC with an ion paring agent having concentration ranging from about 0.01% to about 2% in combination with organic modifier having concentration ranging from about 5% to about 85%.

In an embodiments of the present disclosure most preferably the ion pairing agent and the organic modifier have a concentration ranging from about 0.05% to about 1% and from about 8% to about 65% respectively.

In another embodiment of the present disclosure the ion pairing agent is selected from a group comprising Hexane sulfonic acid, Trifluoro acetic acid, Pentafluoro propanoic acid, Triethyl amine and Heptafluorobutyric acid.

In yet another embodiment of the present disclosure the organic modifier is selected from a group comprising Acetonitrile, Ethanol, Methanol and Isopropyl alcohol.

In still another embodiment of the present disclosure the polypeptide is selected from a group comprising, Insulin Analogue, Eptifibatide and Atosiban.

In still another embodiment of the present disclosure the Insulin analogues are Aspart, Lispro and Glargine.

The present disclosure also relates to a chromatographic process for purification of polypeptide from a mixture having at least one related impurity, said process comprising steps of: a) packing RP-HPLC column with silica (C₄—C₁₈) based resin, equilibrated with about 5% to about 85% organic modifier, b) loading the polypeptide mixture on the column at a flow rate of about 180 cm/hr to about 360 cm/hr, c) washing the column with an ion pairing agent having concentration ranging from about 0.05% to about 1% in combination with the organic modifier having concentration ranging from about 5% to about 85%, and d) performing a linear gradient of about 10% to about 70% for eluting the purified polypeptide from the column.

In an embodiment of the present disclosure the silica resin is preferably C₈.

In another embodiment of the present disclosure the chromatographic purification is carried out at a pH ranging from about 2.5 to about 8.5.

In yet another embodiment of the present disclosure the resin has a particle size ranging from about 5 μto about 40 μ, preferably, from about 7 μto about 20 μ, and most preferably from about 10 μto about 13 μ.

In still another embodiment of the present disclosure the resin bead has a pore size ranging from about 50 Å to about 2000 Å, preferably from about 100 Å to about 500 Å, and most preferably 120 Å.

In still another embodiment of the present disclosure the purity of the polypeptide is ranging from about 90% to about 100%, preferably at least 99%.

The present disclosure further relates to an Insulin analogue obtained by a process as stated above with purity ranging from about 90% to about 100%.

The present disclosure also relates to purified Aspart purified with a purity of at least 98%.

The present disclosure also relates to purified Glargine purified with a purity of at least 99%.

The present disclosure also relates to purified purified Lispro purified with a purity of at least 97%.

The present disclosure also relates to purified a polypeptide obtained by a process as stated above with purity ranging from about 90% to about 100%.

The present disclosure also relates to purified purified Atosiban purified with a purity of at least 99.14%.

The present disclosure also relates to purified purified Eptifibatide purified with a purity of at least 94%.

The disclosure relates to the usage of ion pairing agents such as Triethylamine,

Trifluoro acetic acid, Hexane sulfonic acid, Pentafluoro propanoic acid etc in RP-HPLC preparative linear chromatography of polypeptides. More specifically the disclosure relates to the usage of ion pairing agents through reverse phase preparative linear chromatography of Insulin analogues and peptides

Another object and advantage of the present disclosure is increased purity of the desired protein even after the protein loading was increased.

### DEFINITION OF TERMS

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art.

The term ‘polypeptide’, ‘protein’, ‘peptide’ refers to a polymer of amino acids and does not refer to a specific length of the product; thus, peptides, oligopeptides, and proteins are included within the definition of polypeptide. This term also does not refer to or exclude post-expression modifications of the polypeptide although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present disclosure. In one embodiment, the molecule is a polypeptide or their related analogs or derivatives thereof According to preferred embodiment, the polypeptide is selected from Insulin analogues such as aspart, lispro and glargine. Preferably, the polypeptide is a cyclic peptide. According to another preferred embodiment, the polypeptide is a non-cyclic peptide. In still another preferred embodiment, the polypeptide is selected from the group comprising eptifibatide, and, atosiban .

The term “Insulin analog” is intended to encompass any form of “Insulin” as defined above wherein one or more of the amino acids within the polypeptide chain has been replaced with an alternative amino acid and/or wherein one or more of the amino acids has been deleted or wherein one or more additional amino acids has been added to the polypeptide chain. Insulin analogue is selected from the group comprising Aspart, Lispro and Glargine.

Insulin Aspart is a human analogue that is a rapid-acting, parenteral blood glucose-lowering agent. Aspart is homologous with regular human Insulin with the exception of a single substitution of the amino acid proline by aspartic acid in position B28, and is produced by recombinant DNA technology.

Insulin Glargine differs from human Insulin in that the amino acid asparagine at position 21 on the Insulin A-chain is replaced by glycine and two arginines are added to the C-terminus of the B chain. Insulin Glargine is also a human Insulin analogue that is a rapid-acting, parenteral blood glucose-lowering agent.

Insulin Lispro is a human insulin analogue that is a rapid acting, parenteral blood glucose-lowering agent. Chemically, it is Lys (B28), Pro (B29) human insulin analogue, created when the amino acids at positions 28 and 29 on the Insulin B-chain are reversed.

Atosiban—a synthetic peptide is an inhibitor of the hormones oxytocin and vasopressin. It is an oxytocin receptor antagonist, is effective in the treatment of an acute episode of preterm labor. It is a competitive antagonist of oxytocin at uterine oxytocin receptors and has been developed as a new tocolytic therapy in the treatment of preterm labour. Atosiban also called ADH (Anti-Diuretic Hormone).

Eptifibatide—is an antiplatelet drug of the glycoprotein IIb/IIIa inhibitor class. Its a cyclic heptapeptide derived from a protein found in the venom of the southeastern pygmy rattlesnake. It belongs to the class of the so called arginin-glycin-aspartat-mimetics and reversibly binds to platelets.

The term “chromatography” refers to the process by which an analyte of interest in a mixture is separated from other solutes in a mixture as a result of differences in rates at which the individual solutes of the mixture migrate through a stationary phase under the influence of a moving phase, or in bind and elute processes.

The term “High Performance liquid chromatography”, as used herein, refers to that chromatographic procedure in which the particles (stationary phase) used in the column packing are small (between 3 and 50 microns) and regular with little variation from the selected size. Such chromatography typically employs relatively high (around 500-3500 psi) pressures.

The term “impurity” refers to any product that does not share the same nature as the protein of interest. The impurities are mainly product related. The impurity that has been targeted in the Insulin Aspart crude by using ion pairing agents was Des leader des B arginine Insulin Aspart precursor. This differs from Insulin Aspart by possessing an extension of 5 amino acids at the C-terminal of B chain, the 5 amino acids being Arg-Asp-Ala-Asp-Asp which shows that at acidic pH it has an extra positive charge with respect to Aspart.

“Ion pairing agents” are usually ionic compounds that form ion pairs with oppositely charged ions. Usually ion pairing agents used in RP-HPLC contain a hydrocarbon chain that imparts certain hydrophobicity so that the ion pairing agents can be retained on a reversed-phase column. Typical ion pairing agents capable of forming ion pairs with proteins may be used in the present disclosure which may include Trifluoro Acetic acid (TFA), Hexane sulfonic acid (HSA), Pentafluoro propanoic acid (PFPA), Triethyl amine (TEA), Heptafluorobutyric acid (HFBA), etc.

Specifically this disclosure relates to the use of HSA and TEA in RP-HPLC preparative linear chromatography as exemplified in the preferred embodiments. HSA has a structure composed of a 6 C long hydrocarbon chain with a sulfonic acid (SO₃⁻) functional group. In the acidic pH, essentially below the pI value of a peptide/protein, all the functional amino groups of the basic amino acids such as Lysine, Arginine and Histidine present on the surface of the protein structure would be positively charged due to protonation. SO₃⁻ group of HSA exists in the ionic form even at low pH due to the high electron density on the O-atoms which is attributed to the inductive effect shown by the 6 C long hydrocarbon side chain. The SO₃⁻ group would specifically target the positively charged groups on the protein structure. This interaction between HSA and the peptide would bring about a miniscule and reversible conformational change in the protein 3D structure. This would sometimes bring about a change in the selectivity of some impurities during RP chromatography. HSA is very well known for improving the resolution of protein peaks in the analytical RP chromatography. Whereas in the present disclosure, it has been observed that HSA helps in improving the yields and purity levels in preparative chromatography as well. p Triethyl amine (TEA) as a well known ion pairing agent elutes the protein in the form of tight bands and this property is currently employed in higher loading (preparatory loading) to improve yields and purity of the RP chromatography.

“Organic modifiers” are non-polar solvents that are used in RP chromatography which at lower concentrations assist the analyte molecules to bind to the stationary phase and at higher concentrations elute the same. Various organic modifiers are used in the instant disclosure like acetonitrile, isopropyl alcohol, ethanol, methanol, dimethylformamide etc.

“Desocta” is a product related impurity that gets generated during the trypsinisation step of the process. It is characterized by lacking 8 amino acids towards the C-terminal of the B-chain of Insulin and its analogues.

0.85 RRT, 0.9 RRT, 1.05 RRT and 1.07 RRT are product related impurities that gets generated during reaction intermediates some are generated during process. These impurities are formed because of multiple reaction steps involved in the process of preparation of Atosiban.

0.86 RRT, 1.22 RRT, 1.8 RRT and 2.1 RRT are impurities formed because of multiple reaction step involved in the process of preparation of eptifibatide. They are typically reaction intermediates and some are generated during the process. There can be intermediates which have protected groups that prevent them from converting into the product.

The present disclosure relates to the use of ion pairing agents for obtaining substantially pure Insulin analogs and peptide in the range of 90%-100%. Insulin analogues maybe selected from a group comprising of Aspart, Lispro and Glargine.Protein or peptide is Atosiban and eptifibatide.

It is an object of the present disclosure to use at least 0.05% to 1% (w/v) of an ion pairing agent wherein the process is based upon RP-HPLC.Most preferably ion pairing agent is used in the range of 0.05%-1% (w/v).

In a broad aspect, the present disclosure relates to the use of ion pairing being employed for purifying a protein of interest comprising the steps of:

Equilibration to keep the stationary phase ready to bind to the analyte of interest, followed by loading wherein the crude or the impure material containing the analyte is passed through the stationary phase. The sample is loaded at a flow rate of about 180-360 cm/hr. The gradients used are subject to variation with respect the sample peptide to be purified.

The first step of the process herein involves purifying molecules from mixtures containing them by loading the mixtures on a reversed-phase liquid chromatography column. Preferably, the column is packed with a medium having a particle diameter of about 5-40 μ, more preferably about 10-40 μ, and most preferably about 10-13 μ. Preferably, the column has a pore size of about 100-2000 angstroms, more preferably about 100-500 angstroms. In context of the present disclosure the pore size of the resin packed in the column is 120 angstroms.

This is followed by washing to remove the unbound molecules, eluting the bound analyte from the stationary phase and regeneration to remove any tightly bound molecules that does not elute with the given elution conditions. 5%-65% of various organic modifiers are used for equilibration and washing such as acetonitrile, isopropyl alcohol, ethanol, methanol, dimethylformamide etc. The purification is carried out at a pH in the range of 2.5-8.5.

The medium of the column may be any suitable material, including polymeric-based resin media, silica-based media, or methacrylate media.

One aspect of the present disclosure resides in the use of ion pairing agents in RP-HPLC preparative linear chromatography for obtaining substantially pure proteins and peptides. Typical ion pairing agents capable of forming ion pairs with proteins may be used in the present disclosure which may include Trifluoro Acetic acid (TFA), Hexane sulfonic acid (HSA), Pentafluoro propanoic acid (PFPA), Triethyl amine (TEA), Heptafluorobutyric acid (HFBA), Heptafluorobutyric acid (HFBA), etc.

Specifically this disclosure relates to the use of HSA and TEA in RP preparative linear chromatography as exemplified in the preferred embodiments. HSA has a structure composed of a 6 C long hydrocarbon chain with a sulfonic acid (SO₃⁻) functional group. In the acidic pH, essentially below the pI value of a peptide/protein, all the functional amino groups of the basic amino acids such as Lysine, Arginine and Histidine present on the surface of the protein structure would be positively charged due to protonation. The SO₃⁻ group would specifically target the positively charged groups on the protein structure. As HSA interacts with the surface charges, the hexyl group of HSA would be exposed on the surface of the protein, which would increase its hydrophobicity. The extent of the interaction of HSA with various peptides would bring about a change in their selectivity. HSA is very well known for improving the resolution of protein peaks in the analytical RP chromatography. Whereas in the present disclosure, it has been observed that HSA helps in improving the yields and purity levels in preparative chromatography as well.

Triethyl amine (TEA) also works in the similar way as HSA, TEA ion pairs with the negatively charged moieties on the protein surface and increases the hydrophobicity of the proteins.

Another object of the present disclosure relates to the use of ion pairing agent which helped in changing the selectivity of an impurity which is very closely related to the protein of interest and was a major concern in the purification.

The impurities are mainly product related. The impurity that has been targeted in the Insulin Aspart crude by using ion pairing agents was Des leader des B Arginine Insulin Aspart precursor. This differs from Insulin Aspart by possessing an extension of 5 amino acids at the C-terminal of B chain, the 5 amino acids being Arg-Asp-Ala-Asp-Asp which shows that at acidic pH it has an extra positive charge with respect to Aspart which would be a target for HSA. In Insulin Glargine, HSA helps in separating the Insulin Glargine precursor as it possesses extra positive charges than that of Insulin Glargine.

Yet another aspect of the present disclosure is the increased purity of the desired protein even after the protein loading was increased.

The effective performance of the present disclosure requires the individuation of right combination of the chromatographic matrix to be used, the pH value and the ionic strength of the buffer for efficient purifications.

The pH of the buffer system influences separation combined with hydrophobicity of compounds. The change in pH attributed during different steps of chromatography separation, affect the mobility of compounds in the column.

The foregoing descriptions of specific embodiments of the present disclosure are presented for purposes of illustration and description. They are not intended to be exhaustive or to limit the disclosure to the precise forms disclosed. Various modifications and variations are possible in view of the above teachings. In addition, many modifications may be made to adapt a particular situation, material, composition of matter, process, process step or steps, to the objective, spirit and scope of the present disclosure. All such modifications are intended to be within the scope of the claims appended hereto.

The technology of the instant Application is further elaborated with the help of following examples. However, the examples should not be construed to limit the scope of the disclosure.

The following examples are provided to further illustrate the embodiments of the present disclosure, but are not intended to limit the scope of the disclosure. While they are typical of those that might be used, other procedures, methodologies, or techniques known to those skilled in the art may alternatively be used.

A more complete understanding can be obtained by reference to the following specific examples, which are provided for purposes of illustration only and are not intended to limit the scope of the disclosure.

### EXAMPLES

### Control 1

Crude Insulin Aspart material of purity of ˜75% was diluted 10 times with purified water and IPA was added to a final concentration of 5%. Crude mixture was purified on a Kromasil™ (100 Å-13 μ-C8) column. Mobile phase A was 250 mM Sodium acetate, pH 4.0 and the mobile phase B was Isopropyl alcohol. A Gradient Elution of 15% to 18% of mobile phase B in mobile phase A over 20 column volumes. Separation of desocta Insulin Aspart was observed but desleader desB-arginine Insulin Aspart precursor separation was not achieved. The overall purity came to ˜90%.

### Example 1

Crude Insulin Aspart material of purity of ˜75% was diluted 10 times with purified water and IPA was added to a final concentration of 5%.Crude mixture was purified on a Kromasil™ (100 Å-13 μ-C8) column. Mobile phase A was 1% hexane Sulfonic acid (HSA) (w/v) in 250 mM Sodium acetate, pH 4.0 and the mobile phase B was Isopropyl alcohol. A Gradient Elution of 18%-22% of mobile phase B in mobile phase A over 20 column volumes. The addition of HSA efficiently removed desocta Insulin Aspart and also reduction of desleader desB-arginine Insulin Aspart precursor from a level of 2.77% to less than 0.27% was observed and the overall purity achieved is of ˜97.85%.

### Control 2

Crude Insulin Aspart material of purity of ˜75% was diluted 10 times with purified water and ethyl alcohol was added to a final concentration of 10%. Crude mixture was purified on a Kromasil™ (100 Å-13 μ-C8) column. Mobile phase A was 25 mM ammonium sulphate in 250 mM sodium acetate, pH 4.0 and the mobile phase B was ethyl alcohol. A gradient elution of 26%-32% of mobile phase B in mobile phase A over 20 column volumes. The desleader desB-arginine Insulin Aspart precursor reduced from a level of ˜2% to 1%. The desocta Insulin Aspart impurity was removed completely and the monoglycosylated Insulin Aspart was reduced from ˜3% to 1.3%. The overall purity achieved is of ˜94%

### Example 2

Crude Insulin Aspart material of purity of ˜75% was diluted 10 times with purified water and ethyl alcohol was added to a final concentration of 10%. Crude mixture was purified on a Kromasil™ (100 Å-13 μ-C8) column. Mobile phase A was 1% Hexane Sulfonic acid (HSA) (w/v) in 25 mM ammonium sulphate in 250 mM sodium acetate, pH 4.0 and the mobile phase B was ethyl alcohol. A gradient elution of 25%-35% of mobile phase B in mobile phase A over 20 column volumes. The addition of HSA reduced desleader desB-arginine Insulin Aspart precursor from a level of ˜3% to less than 0.3%. The desocta Insulin Aspart was completely removed and the monoglycosylated Insulin Aspart levels was reduced from ˜2% to 0.3%. The overall purity achieved is of ˜98%

### Control 3

Crude Insulin Aspart material of purity of ˜67% was diluted 10 times with purified water and methyl alcohol was added to a final concentration of 10%. Crude mixture was purified on a Kromasil™ (100 Å-13 μ-C8) column. Mobile phase A was 25 mM ammonium sulphate in 250 mM Ammonium acetate, pH 4.0 and the mobile phase B was methyl alcohol. A gradient elution of 45%-55% of mobile phase B in mobile phase A over 20 column volumes. The desleader desB-arginine Insulin Aspart precursor reduced from a level of ˜2% to less than 1%. The monoglycosylated Insulin Aspart impurity did not show any significant reduction. The reduction was from 2% to 1.5%. The desocta Insulin Aspart reduced from ˜10% to less than ˜2%. The overall purity achieved is of ˜89%

### Example 3

Crude Insulin Aspart material of purity of ˜67% was diluted 10 times with purified water and methyl alcohol was added to a final concentration of 10%. Crude mixture was purified on a Kromasil™ (100 Å-13 μ-C8) column. Mobile phase A was 1% Hexane Sulfonic acid (HSA) (w/v) in a 25 mM ammonium sulphate in 250 mM Ammonium acetate, pH 4.0 and the mobile phase B was methyl alcohol. A gradient elution of 55%-65% of mobile phase B in mobile phase A over 20 column volumes. The addition of HSA purified desleader desB-arginine Insulin Aspart precursor from a level of ˜2% to less than 1%. The monoglycosylated Insulin Aspart impurity was reduced from 3% to less than 0.5%. The desocta Insulin Aspart reduced from ˜10% to less than ˜2%. The overall purity achieved is of ˜92%

### Example 4

Crude Insulin Aspart material of purity of ˜67% was diluted 10 times with purified water and methyl alcohol was added to a final concentration of 10%. Crude mixture was purified on a Kromasil™ (100 Å-13 μ-C8) column. Mobile phase A was 1% hexane sulfonic acid (HSA)(w/v) in 25 mM ammonium sulphate in 250 mM ammonium acetate, pH 4.0 and the mobile phase B was methyl alcohol. A gradient elution of 20%-50% of mobile phase B in mobile phase A over 5 column volumes followed by 55%-65% of mobile phase B in mobile phase A over 20 column volumes. The addition of HSA purified desleader desB-arginine Insulin Aspart precursor from a level of ˜2% to less than 1% and monglycosylated impurity from ˜3% to 0.6%. The desocta Insulin Aspart reduced from ˜10% to less than ˜2%. The overall purity achieved is of ˜92%.

### Example 5

Crude Insulin Aspart material of purity of ˜67% was diluted 10 times with purified water and methyl alcohol was added to a final concentration of 10%. Crude mixture was purified on a Kromasil™ (100 Å-13 μ-C8) column. Mobile phase A was 1% Hexane Sulfonic acid (HSA) (w/v) in 25 mM ammonium sulphate in 250 mM ammonium acetate, pH 3.5 and the mobile phase was methyl alcohol. A gradient elution of 55%-60% of mobile phase B in mobile phase A over 20 column volumes. The addition of HSA purified monglycosylated Insulin Aspart from a level of ˜3% to less than 0.4%. The desocta Insulin Aspart reduced from ˜10% to less than ˜1%. The overall purity achieved is of ˜90%.

### Control 6

Partially purified Insulin Glargine material of purity of ˜94% was diluted 3 times with purified water and methyl alcohol was added to a final concentration of 10%. Crude mixture was purified on a Kromasil™ (100 Å-13 μ-C8) column. Mobile phase A was 100mM ammonium sulphate, pH 4.0 and the mobile phase B was methyl alcohol. A gradient elution of 51%-59% of mobile phase B in mobile phase A over 20 column volumes. The Insulin Glargine precursor levels were reduced from ˜2.0% to 1.2% and DesB32-R Insulin Glargine was not reduced. The overall purity achieved is of ˜97.5%.

### Example 6

Partially purified Insulin Glargine material of purity of ˜94% was diluted 3 times with purified water and methyl alcohol was added to a final concentration of 10%. Crude mixture was purified on a Kromasil™ (100 Å-13 μ-C8) column. Mobile phase A was 0.5% Hexane Sulfonic acid (HSA)(w/v) in 100 mM ammonium sulphate, pH 4.0 and the mobile phase B was methyl alcohol. A gradient elution of 51%-59% of mobile phase B in mobile phase A over 20 column volumes. The addition of HSA purified Insulin Glargine precursor from a level of ˜2.5% to less than 0.8% and DesB32-R from 0.7% to less than 0.2%. The overall purity achieved is of ˜99.2%.

### Example 7

Partially purified Insulin Lispro material of purity of ˜77% was diluted 3 times with purified water and acetonitrile was added to a final concentration of 10%. Crude mixture was purified on a Kromasil™ (100 Å-13 μ-C8) column. Mobile phase A was 1% Triethyl amine (TEA) (v/v) in 20 mM magnesium chloride and 100 mM Tris, pH 8.5 and the mobile phase B was acetonitrile. A gradient elution of 24%-30% of mobile phase B in mobile phase A over 25 column volumes. The addition of TEA purified desleader desB-Arginine Insulin Lispro precursor level of 15% to less than 1%. The overall purity achieved is of ˜97.5%.

### Control 8

Atosiban crude after one step of purification is at a purity of ˜95%. The elution pool of first step is diluted in such a way as to get final solvent concentration to 5%. The load is then purified on a Daiso (120 Å-10 μ-C8) column. Mobile phase A was 50 mM acetic acid and mobile phase B was Acetonitrile. A gradient elution of 9% to 12% of mobile phase B in mobile phase A over 15 column volumes followed by 12 to 17% mobile phase B in mobile phase A over 10 column volumes was carried out. The purity achieved was ˜96% showing complete removal of only 1.05 RRT impurities. The 0.85 RRT and 0.9 RRT impurity did not show any reduction.

### Example 8

Atosiban crude after one step of purification is at a purity of ˜95%. The elution pool of first step is diluted in such a way as to get final solvent concentration to 5%. The load is then purified on a Daiso (120 Å-10 μ-C8) column. Mobile phase A was 0.05% hexane sulphonic acid (HSA) (w/v) in 50 mM acetic acid and mobile phase B was Acetonitrile. A gradient elution of 10% to 17% of mobile phase B in mobile phase A over 25 column volumes was carried out. The addition of HSA helped in increasing the purity to 98.6% showing complete removal of 0.85 RRT, 0.90 RRT, 1.05 RRT and 1.07 RRT impurities.

### Control 9

Atosiban crude after one step of purification is at a purity of ˜95%. The elution pool of first step is diluted in such a way as to get final solvent concentration to 5%. The load is then purified on a Daiso (120 Å-10 μ-C8) column. Mobile phase A was 50 mM phosphate buffer at pH 8.0 and mobile phase B was Acetonitrile. A gradient elution of 18% to 24% of mobile phase B in mobile phase A over 25 column volumes was carried out. The purity achieved was 98.5%. The 0.97 RRT reduced from ˜1.7% to ˜0.6%. The 0.95 RRT impurity was not removed, the 1.05 RRT and 1.07 RRT impurities reduced from ˜0.8% to ˜0.3%.

### Example 9

Atosiban crude after one step of purification is at a purity of ˜95%. The elution pool of first step is diluted in such a way as to get final solvent concentration to 5%. The load is then purified on a Daiso (120 Å-10 μ-C8) column. Mobile phase A was 0.2% Triethyl amine (TEA) (v/v) in 50 mM phosphate buffer at pH 8.0 and mobile phase B was Acetonitrile. A gradient elution of 20% to 24% of mobile phase B in mobile phase A over 25 column volumes was carried out. The addition of TEA helped in increasing the purity to 99.14% showing complete removal of 0.95 RRT, 0.97 RRT impurities and 50% reduction in 1.05 RRT and 1.07 RRT impurities.

### Example 10

Crude Eptifibatide is at a purity of ˜58%. Load is prepared by dissolving the crude in 50 mM acetic acid and 5% acetonitrile. The load is then purified on a Daiso (120 Å-10 μ-C8) column. Mobile phase A was 0.1% Trifluro acetic acid (TFA) (v/v) and mobile phase B was Acetonitrile. A gradient elution of 8% to 14% of mobile phase B in mobile phase A over 25 column volumes was carried out. The addition of TFA helped in increasing the purity to 94% showing complete removal of 0.86 RRT, 1.22 RRT, 1.8 RRT and 2.1 RRT impurities.

Preferred embodiments of the present disclosure have been disclosed. A person of ordinary skill in the art would realize, however, that certain modifications would come within the teachings of this disclosure. Therefore, the following claims should be studied to determine the true scope and content of the disclosure.

We claim:

1. A chromatographic process for purification of insulin analogue, atosiban or eptifibatide from a mixture having at least one related impurity, at a pH ranging from about 2.5 to about 8.5, said process comprising steps of:
packing RP-HPLC column with silica (C₄—C₁₈) based resin, equilibrated with about 5% to about 85% organic modifier;
loading the insulin analogue, atosiban or eptifibatide mixture on the column at a flow rate of about 180 cm/hr to about 360 cm/hr;
washing the column with an ion pairing agent having concentration ranging from about 0.05% to about 1% in combination with the organic modifier having concentration ranging from about 5% to about 85%, at pH ranging from about 2.5 to about 8.5 and;
performing a linear gradient of about 10% to about 70% for eluting the purified insulin analogue, atosiban or eptifibatide from the column.

2. The process as claimed in claim 1, wherein the insulin analogue is selected from a group comprising Aspart, Lispro and Glargine or any combination thereof

3. The process as claimed in claim 1, wherein the silica resin is preferably C₈.

4. The process as claimed in claim 1 wherein, the resin has a particle size ranging from about 5 μto about 40 μ, preferably, from about 7 μto about 20 μ, and most preferably from about 10 μto about 13 μ.

5. The process as claimed in claim 1, wherein the resin bead has a pore size ranging from about 50Å to about 2000Å, preferably from about 100Å to about 500Å, and most preferably 120Å.

6. The process as claimed in claim 1, wherein the ion pairing agent is selected from a group comprising Hexane sulfonic acid, Trifluoro acetic acid, Pentafluoro propanoic acid, Triethyl amine and Heptafluorobutyric acid.

7. The process as claimed in claim 1, wherein the organic modifier is selected from a group comprising Acetonitrile, Ethanol, Methanol and Isopropyl alcohol.

8. The process as claimed in claim 1, wherein purity of the insulin analogue, atosiban or eptifibatide is ranging from about 90% to about 100%, preferably at least 99%.

9. An Insulin analogue, atosiban, eptifibatide obtained by a process as claimed in claim 1 with purity ranging from about 90% to about 100%.

10. Purified Aspart purified with a purity of at least 98%.

11. Purified Glargine purified with a purity of at least 99%.

12. Purified Lispro purified with a purity of at least 97%.

13. Purified Atosiban purified with a purity of at least 99.14%.

14. Purified Eptifibatide purified with a purity of at least 94%.
